SEQUENCE LISTING (8 sequences)

SEQ ID NO: 1 - protein, 482 aa, homo sapiens
MAALMTPGTGAPPAPGDFSGEGSQGLPDPSPEPKQLPELIRMKRDGGRLSEADIRGFVAAVVNGSAQGAQIGAMLMAIRLRGMDLEETSVLTQALAQSGQQLEWPEAWRQQLVDKHSTGGVGDKVSLVLAPALAACGCKVPMISGRGLGHTGGTLDKLESIPGFNVIQSPEQMQVLLDQAGCCIVGQSEQLVPADGILYAARDVTATVDSLPLITASILSKKLVEGLSALVVDVKFGGAAVFPNQEQARELAKTLVGVGASLGLRVAAALTAMDKPLGRCVGHALEVEEALLCMDGAGPPDLRDLVTTLGGALLWLSGHAGTQAQGAARVAAALDDGSALGRFERMLAAQGVDPGLARALCSGSPAERRQLLPRAREQEELLAPADGTVELVRALPLALVLHELGAGRSRAGEPLRLGVGAELLVDVGQRLRRGTPWLRVHRDGPALSGPQSRALQEALVLSDRAPFAAPSPFAELVLPPQQ

SEQ ID NO: 2 - protein, 482 aa, homo sapiens [sequence omitted]

SEQ ID NO: 3 - protein, 48 aa, homo sapiens
LAPRAPGRPRAQRPAEPRPAGGARTLRPRAIRRPLALRRARSAAAAIK

SEQ ID NO: 4 - protein, 48 aa, homo sapiens
LAPRAPGRPRAQRPAEPRPAGGARTLRPHAIRRPLALRRARSAAAAIK

SEQ ID NO: 5 - protein, 11 aa, homo sapiens
RPRAIRRPLAL

SEQ ID NO: 6 - protein, 11 aa, homo sapiens
RPHAIRRPLAL

SEQ ID NO: 7 - DNA, 1600 bp, homo sapiens
gccccgccgccggcagtggaccgctgtgcgcgaaccctgaaccctacggtcccgacccgcgggcgaggccgggtacctgggctgggatccggagcaagcgggcgagggcagcgccctaagcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtggaggggctgtccgctctggtggtggacgttaagttcggaggggccgccgtcttccccaaccaggagcaggcccgggagctggcaaagacgctggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcgggggcgccctgctctggctcagcggacacgcggggactcaggctcagggcgctgcccgggtggccgcggcgctggacgacggctcggcccttggccgcttcgagcggatgctggcggcgcagggcgtggatcccggtctggcccgagccctgtgctcgggaagtcccgcagaacgccggcagctgctgcctcgcgcccgggagcaggaggagctgctggcgcccgcagatggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtgggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcgaaa

SEQ ID NO: 8 - DNA, 5845 bp, homo sapiens
gatattcggtaccccatatccggatctcaaatcccaaaccccgaaccccacggggctttgataaatcgtggctcagactccccactagtcccaggaccccatctcgggtacccaccaggctcccacgcagttctagccccccacacccttgatccgccccgcaggcaggtacttcccggagcgagcggggaacgcgacgtaccccagtgcgcctcggcacaccattgctccccgaaactggggtgtccaggcggaacagcagagcccaggtgaggtcagaacggcccatcccagaactgtgggccttcccactcgagaccggggaccgccctccgggagctgggaccaccctgcgcctgtccgcggagacccactacccccgagccctgcctcctccccaggtcccgcggcctatacggtgccctcgctcttgggtccgcgcgtcatcggcaaagtctccgccccaacttgctccatctacggccgcagagcggctggcagtttcttcgaggacctcagcaaggtgggggaggggccggggcggacgcagggggtccctggtccgcggcagtggaggcggcagccagcaccctctgccctctcgcagaccccgggcccctgcgcctatcaggtcgtgagtccaggggtctacaagtcccgggccccccagttcacgattctggcgcggacttcgctcccccaagacaacactcggaagccagggcccgcggcctacaacgtggatcaggtggcctggagcccagggtcaagggtcagagtcaggagagtggggagggcctgaggtcggagtgatgggatcagagtccccgggggtccaggggtcccggcgcggagaggatgccggccccgcgaggtcagcggtgtctccgggcccgcagcaccggaagccccgcggctggagtttcgggatccggcactcggactacctggccccgctggtgaccgacgcggacaactgacccgccaggcgggagcggccccacacgtgtttgcttaaagtctgcgagtccgcatcgtgtccgcctctctctctctctctctgcgcgtcctggcgcaaggcctggggtggagccacggctggggccgtgtcccaactccgaacccagcggggcggggcccgagcgtcgggcgaggccgggaccccagcgctgcgccgcgtccgaacgtcgagaccccaccgagggcgggagggggactctcgggagccacagacgcccgagacccacgccgggcgggaccggccagggatcacccccgccgacggccccgggccccgacggcccggaagttccgcgtgtccgggggcaccgggggattggccggggcgcggcgtgcaaggcttcccgggggcggcgactgccgagctccgccctccaggcggccccacccgcctgccgtcctggggcgccgccgccccgccgccggcagtggaccgctgtgcgcgaaccctgaaccctacggtcccgacccgcgggcgaggccgggtacctgggctgggatccggagcaagcgggcgagggcagcgccctaagcaggtacgggcggggctcaagtcgcgaggcggggaagcgggaggcagacacggacgagggcgacacagacacgggaccgaggggcggacaccggagagacacgggaaaggggtcgggacaggagcacgtggctcagacaccgacgccgggaggccgcagaccccggacgtgtcaggcatccccgcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggtgcgtggggagggttgggcgttcctgaccccgactgggaggtcagcccgagagactttgggtccctgggggtgcgacggtgccccactaccagcaccggccccagggtgccccaccgctgtgggctgccaccctcacgcgtacccccacataccaggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggttagaaaccacctcctttccagacgggagcctataccgcacatgcagcaaccagtccatccacaggcagctcccaacctcaagcctggcccaaagcctccaagaccctaccaaggcttctccccaccctgctccccagcacagttctccccaccccgttccccagcacagcgcttggggcccctctggctccagaccaggccccttggagcaggaaaaagatccactgatggaattcagacccctttccccttgggtccccagacagctcccccaagggaggagctgaggacttccctccctctgccccaagccttgtttccccaaggagaggtaccaacctcctcccctactgacacttctcaaccaagaaaacttcctttccattccctcaccagctgggcacccctatagctgcttaaatactttccaaatccagctgcactcctagccagggaaggtgaagggatgcacagaggtgggggaggggtactgtgcagggtactcagcatccctgaccaccaggtgccaatgatcagcggacgtggtctggggcacacaggaggcaccttggataagctggagtctattcctggattcaatgtcatccagagcccagagcaggtacggggcgccacggatcagtcattgatccaggttgatgatccagaccctggccagaatcactaaaagatcactggtggatcattagggtcactaatgagaacactggtcaaggttactcatgagtcactgggcctgggccgaaatcatcagtggaactttgattaggatcataaaatgggaagttggtcaaaatcacagatggctggcggggcacggtggctcacacctgtagtcctagcacttggggaggccgaagagggcagatcccttgaacccaggagttcaaaaccagcctggataacaaggcaaaaccccatctctacaaaatagttcgctgcgtgtggtggtgcacgcatgtggttccagctactcaggaggctgaggcaggaggatcacttgagcctgggaggtctaggctgcagtgagccgggacgatgccactgcactccagcctgggcaacagagtgagaccctgtcccagcactctgggaggcagaggagcccagttggagatcagcctgggtaatatagtgaaacttgatctctacaaaaaaaagaagaaaaaaaaaagccgcgtgtggtggtgcgcacctgtagtcccagctactgggaagctgaggtgggaggatcacttaagcccaggaggcagaggtcacaatgagccgaaattgtgccaactgcactccagcctgggcaacagaggaagactcttcacagaaaaaaaaaaaaaaaaaaagctgctaagtcatttaccataagtcactgagaacaggggatgtctgaccagatgcaagtgctgctggaccaggcgggctgctgtatcgtgggtcagagtgagcagctggttcctgcggacggaatcctatatgcagccagagatgtgacagccaccgtggacagcctgccactcatcacaggtgacctgactccatggcctgcttctgcatgttcacaggctcctgacctccaaactcaagtcaagggcctctcgttaggagttacccgtcacctgaccgtgtgcccccctacccccatcacaagatgcctgaccaccaccatgtgggtggcctgatactcaacccaccaggtgctgccacccccataataagggacttgaccctcaatgctcagggcccctgaccccaaagtcggcatccccgaactctcccaagaagctccaggttctccattgtctccaacctcctctgcctcccccaaagcctccattctcagtaagaaactcgtggaggggctgtccgctctggtggtggacgttaagttcggaggggccgccgtcttccccaaccaggagcaggcccgggagctggcaaagacgctggtgagcggtgtggcctttccctgggcaagcgtcttgatgcgggcccagcctacccttcacccctcccgtccccactgcctccctccactcagcagtcctgcctaaccccagtcccaccctcttctgcccgaagtccctccctccttcacggcttcctaacctgctgtgactttagaggtcaaggctggcccggcctggacctggggaagccctctgtggggttcctgccccagaccaagtacaagttcctcctggccccatggcgaggtgtcgcacttcactcgtgtctcttccccaccccaatccttccctgacttcatgctggggggctggcaacccaccctgcagcaggggctggagttcgaccaagaaccggctgcagaaggccccgccatggggggtccacgctgagcctcctctccgcaggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcggtgagggggacggggtgtaggggagcggaggcggcggggggtgcttcccgctggggccgccccgacccggccgcgcctaagacccgtccccgcccgcagggggcgccctgctctggctcagcggacacgcggggactcaggctcagggcgctgcccgggtggccgcggcgctggacgacggctcggcccttggccgcttcgagcggatgctggcggcgcagggcgtggatcccggtctgacccgagccctgtgctcgggaagtcccgcagaacgccggcagctgctgcctcgcgcccgggagcaggaggagctgctggcgcccgcagatggtgagcgtcgggggagtccccgtccttccgcctccgccatccccttcccttcccgaggccccgccccttcccgagcccgcgcctctcagcccctctccccgcaggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtggtgagcgccgcccccgccctgctggccccgcacccccgcccagctccggccgcgcggcctctaacagcccctcgctctgcagggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcgaaaccttgtcagtgcttgggcgggagcggagggatccagggctgcggaggcgggggccgtctcgatgaacacgtgacccccggcgggctccgccttccgcgcacgcgctgagagcctgtcagcggctgcgcccgtgtgcgcatgc

(19) United States
(12) Patent Application Publication
(10) Pub. No.: US 2009/0004162 A1
(43) Pub. Date: Jan. 1, 2009

(54) Hecgf-1 Related Polymorphisms and Applications Thereof

(76) Inventors: Elisabeth Helena Slager, Oegstgeest (NL); Johan Herman Frederik Falkenburg, Oegstgeest (NL)

Correspondence Address: MORGAN LEWIS & BOCKIUS LLP, 1111 PENNSYLVANIA AVENUE NW, WASHINGTON, DC, 20004, US

(21) Appl. No.: 11/993,886

(22) PCT Filed: Jun. 29, 2006
(86) PCT No.: PCT/NL2006/050157

§ 371 Date: Jun. 24, 2008

(30) Foreign Application Priority Data
Jun. 29, 2005 (EP) 05105798.2

(51) Int. Cl. A61K 35/12; C07K 14/435; C12N 5/00; C07H 21/04; C12N 5/02; C07K 16/28; A61K 38/17; A61K 31/7088

(52) U.S. Cl. 424/93.21; 530/324; 530/350; 435/325; 536/23.5; 514/12; 514/44; 530/387.3; 530/387.1; 530/387.9; 435/377

(57) ABSTRACT

The invention identifies tumor-associated antigens that may be used for immunotherapy of malignancies in patients having undergone an allogeneic stem cell transplantation, whereby the therapy is mediated by induction of a graft versus tumor immune response. The invention discloses minor histocompatibility antigens encoded by polymorphisms in reading frames present in the hECGF-1 gene. The invention provides peptides comprising polymorphic minor histocompatibility binding peptides or fragments, which may be in the context of an MHC molecule. The invention also provides T cell receptors and T lymphocytes capable of binding to these minor histocompatibility antigens, preferably in the context of MHC molecules. The molecules and cells of the invention can be used for treatment of subjects and manufacture of medicaments for the treatment of subjects suffering from malignancies expressing the hECGF-1 protein.

### FIELD OF THE INVENTION

The current invention relates to the field of medicine, in particular to the fields of stem cell transplantations, immunotherapy and prophylaxis of neoplastic disease.

### BACKGROUND OF THE INVENTION

The identification of tumor-associated antigens and the growing understanding of tumor-specific immune responses provide new possibilities to develop cellular immunotherapy as a strategy for the treatment of cancer. However, the results of many clinical trials have been disappointing since clinical responses were observed in only a limited number of patients (1,2). Vaccination protocols have not led to improvement in overall survival of cancer patients. The main impediment of these vaccination strategies is that in most cases non-mutated self-proteins were targeted. In patients, T cells specific for these self-antigens are probably anergic, tolerized or of low affinity due to peripheral or central selection processes.

High-avidity T cell responses capable of eradicating hematological tumors can be generated in an allogeneic setting. In hematological malignancies, allogeneic HLA-matched hematopoietic stem cell transplantation (SCT) provides a platform for allogeneic immunotherapy due to the induction of T cell-mediated graft-versus-tumor (GVT) immune responses. The clinical potency of the GVT reactivity has been demonstrated by the induction of complete remissions by administration of donor lymphocyte infusion (DLI) in patients with relapsed leukemia after allogeneic SCT (3-5). Immunotherapy in an allogeneic setting enables induction of effective T cell responses due to the fact that T cells of donor origin are not selected for low reactivity against self-antigens of the recipient. Therefore, high-affinity T cells against tumor- or recipient-specific antigens can be found in the T cell inoculum administered to the patient during or after SCT. The main targets of the tumor-reactive T cell responses are polymorphic proteins for which donor and recipient are disparate, designated minor histocompatibility antigens (mHag) (6), or overexpressed proteins like proteinase-3 (7).

Minor histocompatibility antigens (mHag) are epitopes comprised in immunogenic peptides derived from cellular proteins containing differential amino acid compositions due to polymorphisms in the genome of a subject. Disparity in mHag between donor and recipient of allogeneic HLA-matched stem cell transplantation (SCT) leads to stimulation of mHag-specific CD4⁺ and CD8⁺T cells that are involved in alloimmune responses, including non desirable graft rejection or graft-versus-host disease (GVHD) and desirable graft-versus-tumor (GVT) including graft-versus-leukemia/lymphoma (GVL) reactivity.

Induction of GVT reactivity may coincide with the development of GVHD, especially when immune responses are directed against mHags that are broadly expressed in various tissues. GVT can be separated from GVHD by induction of T cells against target structures specific for or overexpressed in tumor cells. In addition, antigens for which expression is restricted to cells of hematopoietic origin, like HA-1 (8), HA-2 (9,10) and BCL2A1 (11), may serve as specific targets for GVT. T cells specific for these antigens will destroy both malignant and normal cells of the hematopoietic system of recipient origin. Because after allogeneic SCT hematopoietic stem cells have been replaced by donor-derived cells that are not recognized by these T cells, normal donor hematopoiesis in the patient will not be affected.

Appropriate antigens for tumor-associated T cell responses that play a role in vivo can be identified by analysis of patients with good clinical responses after allogeneic hematopoietic SCT. Characterization of the target structures of the T cell responses in patients with relapsed hematological cancers that respond to DLI with no or limited GVHD may result in the identification of clinically relevant tumor-specific targets for immunotherapy of cancer.

Several mHag are derived from genes located at the Y chromosome (H-Y antigens) that contain polymorphic amino acids compared to their homologues encoded by the X chromosome (12-18) and U.S. Pat. No. 6,521,598. These male-specific mHag have been shown play a role in sex-mismatched, HLA-matched allogeneic SCT (19). Polymorphisms in autosomal genes have also been described to encode mHag. Some of these mHag, like HA-3 (20), HA-8 (21) and UGT2B17 (22) display broad tissue distributions, whereas the expression of other mHags, like HA-1 (8) and WO 03/047606, HA-2 U.S. Pat. No. 5,770,201 (9,10), HB-1 (23) and BCL2A1 (11), are restricted to cells of hematopoietic origin. T cell responses induced against hematopoiesis-restricted mHag may favour GVL reactivity and reduce the development of GVHD. However, it has also been described that mismatches of hematopoiesis-specific mHag, like HA-1 are correlated with GVHD, probably due a multistep development of GVHD in which T cell responses against mHag-positive antigen-presenting cells (APC) cause a local inflammation that leads to induction of T cell responses to broadly-expressed mHag.

Several mechanisms of differential expression or recognition of mHags have been described. A single nucleotide polymorphism (SNP) in the gene may result in an amino acid substitution in the protein. The polymorphism might affect a TCR contact residue as demonstrated for HB-1 (23) and BCL2A1 (11). Polymorphisms might affect splicing of the messengers or can cause changes in the antigen processing pathway, including proteasomal cleavage, like demonstrated for HA-3 (20) and TAP translocation as shown for HA-8 (21). Next to amino acid difference that affect antigen processing, presentation or recognition, differential mHag expression has also been described to result from deletion of a member of a multi gene family (22).

The aim of the current invention is to identify new mHags with improved properties for the treatment of neoplastic disease within the context of allogeneic stem cell transplants.

### SUMMARY OF THE INVENTION

The invention achieves this aim by the use of new minor histocompatibility antigens and CTL clones specific for these mHag's, isolated from a patient treated with donor lymphocyte infusion (DLI) as a curative treatment for relapsed or persistent malignancies after HLA-matched allogeneic SCT. CTL lines obtained from patients suffering from neoplastic disease were used for characterization of novel mHags that are relevant for control of the malignancy after transplantation and, most importantly, in the absence of severe side effects, in particular a severe graft versus host response.

In this specification, we describe the identification of novel minor histocompatibility antigens in the human ECGF-1 gene. An hECGF-1 gene encoded mHag was recognized by a CTL clone, the HLA-B7-restricted CTL clone RDR173, also designated LB-ECGF-1H, which was isolated from a human patient who was successfully treated for relapsed multiple myeloma with DLI. Using cDNA expression cloning, the mHag identified was encoded by a SNP in the hECGF-1 gene. The epitope recognized by CTL clone RDR173 is derived from translation in an alternative ORF in the ECGF-1 gene due to a non-conventional translation mechanism. Minor histocompatibility antigens encoded by the hECGF-1 gene may not only provide a suitable alternative to known mHags. Due to its biological properties and medical implications, the ECGF-1 gene and an immune response against it provides unique opportunities for improved treatment of neoplastic disease in the context of allogeneic stem cell transplants. The ECGF-1 gene is predominantly expressed in cells of hematopoietic origin and overexpressed in various solid tumors (24-28), playing a critical role in tumor vascularization (24-27) and apoptotic responses (29-33). Hence it constitutes an important target for immunotherapy of cancer. The identification of this new mHag may be successfully exploited to be used for new methods and means to treat both hematological malignancies and solid tumors.

### DETAILED DESCRIPTION OF THE INVENTION

ECGF-1 gene (SEQ ID No. 8) encodes a thymidine phosphorylase enzyme (EC 2.4.2.4 IUBMB nomenclature) and is also referred to as (blood platelet-derived) endothelial cell growth factor. Interestingly, expression of human ECGF-1 has also been shown in many solid tumors, like breast carcinoma (24) renal cell carcinoma (25), colorectal cancer (28), cervical carcinoma (27) and ovarian carcinoma (26). Expression is detected in neoplastic, stromal and/or infiltrating cells, but the cell types that were positive for ECGF-1 varied, even in tumors of same histological origin. ECGF-1 expression is positively correlated with microvesseldensity (24,25) and appears to be an unfavorable prognostic factor in some tumors (25,28). The correlation of ECGF-1 with tumor progression and worse prognosis can be explained by the angiogenetic activity of ECGF-1. The angiogenic effect of ECGF-1 produced by tumor cells and tumor-infiltrating monocytes has recently been described to be mediated via 2-deoxyribose, a metabolite produced by the thymidine phosphorylase activity of ECGF-1, that forms a chemotactic gradient for endothelial cells (34). Angiogenesis is essential for tumor growth since it prevents that supply of oxygen and nutrients to tumor cells is limited to diffusion. The angiogenic role of ECGF-1 is exploited by the current invention to target an immune response to solid and vascularised tumors and in methods to inhibit angiogenesis in a tumor.

Another role for ECGF-1 in tumor progression has been found. Although the exact mechanism has to be elucidated, expression of ECGF-1 by tumor cells seems to enhance resistance to apoptosis induced by hypoxia (29,30), Fas (31,32) or cisplatin (33). Immunotherapy of malignancies will be successful when immune responses are directed against proteins essential for survival of the tumor cells. Since tumor cells are genetically instable, antigen-loss variants may arise that will escape T cell responses. However, when the antigen is essential or beneficial for survival, these antigen-loss variants are less likely to grow out. Although ECGF-1 expression is not essential for cell survival, it seems to be correlated with tumor progression and poor prognosis due to its angiogenic and anti-apoptotic effects. For all the above reasons, ECGF-1 might be an interesting target for immune responses against solid tumors as well, since tumor cells escaping ECGF-1-specific T cells due to shut down or loss of antigen expression may gain susceptibility to apoptosis.

Disease causing mutations are yet another aspect of hECGF-1. Thymidine phosphorylase deficiency caused by mutations in the ECGF-1 gene results in a human mitochondrial disorder MNGIE, mitochondrial neurogastrointestinal encephalomyopathy (35). Homozygous or compound-heterozygous mutations in the ECGF-1 gene specifying thymidine phosphorylase (TP), located on human chromosome 22q13.32-qter are responsible for MNGIE, an autosomal recessive disorder of intergenomic communication. The disorder is defined clinically by 1) severe gastrointestinal dysmotility; 2) cachexia; 3) ptosis, opthalmoparesis, or both; 4) peripheral neuropathy; and 5) leukoencephalopathy. TP deficiency results in multiple deletions of skeletal muscle mitochondrial DNA (mtDNA), which have been ascribed to a defect in communication between the nuclear and mitochondrial genomes. A graft versus host and/or tumor according to this invention does not lead to a similar ECGF-1 deficiency and syndrome. No signs of ECGF-1 deficiency were observed in the patient from whom the ECGF-1 mHag specific T-cell clone was obtained, over several years after remission of the tumor. The development of the MNGIE syndrome requires ECGF-1 deficiency from gestation to adult development.

T cell responses against mHag that are preferentially restricted in expression to the hematopoietic tissues are attractive candidates to be used for cellular immunotherapy in the context of allogeneic SCT and/or donor lymphocyte infusions, compared to T cell responses against broadly expressed mHag due to reduced likelihood of development of severe GVHD. Gene expression profiling and real-time RT-PCR demonstrates a relatively hematopoietic-specific expression of the ECGF-1 gene with high levels of expression in CD14⁺ cells. However, ECGF-1 is also detected in normal tissues, like liver and lung. An immunohistochemical analysis demonstrated that ECGF-1 is predominantly expressed by macrophages in these tissues, although moderate or weak expression in e.g. stromal cells is also detected (36). Low levels of ECGF-1 expression may have implications for the development of GVHD reactivity when the ECGF-1 mHag will be used in immunotherapy of hematological malignancies, but appropriate steps can be taken to minimize these problems, such as immune suppression or tolerization methods known to the skilled physician. Furthermore, the observation that no severe GVHD was observed in the patient from whom the ECGF-specific CTL clone was isolated, demonstrates that ECGF-1-specific T cell clones may be useful for therapeutic purposes.

The current invention pertains to the identification and practical applications of new minor histocompatibility antigens and the invention provides in a first embodiment polymorphic minor histocompatibility antigens encoded by the hECGF-1 gene (SEQ ID No. 8). A first polymorphic mHag antigen or epitope, recognized by a CTL clone isolated from a multiple myeloma patient successfully treated with DLI after allogeneic HLA-matched SCT, is encoded by a SNP at nucleotide 1526 in the ECGF-1 cDNA (SEQ ID No. 7), affecting the amino acid at position 465 in the ECGF-1 protein (SEQ ID No. 1, alternative allele SEQ ID No. 2). The mHag arises from translation in an alternative open reading frame (ORF) instead of the primary ECGF-1 ORF. The G-to-A transition results in a Arg-to-His substitution in an alternatively translated peptide (depicted in FIG. 4 and SEQ ID No. 3, alternative allele SEQ ID No. 4). Although binding to HLA B7 is comparable for both peptide variants, the Arginine containing peptide variant was not recognized by CTL clone upon pulsing HLA-B7 positive target cells, illustrating that the SNP affects a TCR contact residue. The HLA-B7 binding peptides are shown in SEQ ID Nos. 5 and 6.

Since the SNP at pos 1526 and other nearby located SNP's, for instance at position 1545, also lead to amino acid substitutions in the primary ECGF-1 ORF (SEQ ID No. 7) and in its alternative ORFs, in polypeptides predicted or shown to be processed by the proteasome and capable of binding to one or more HLA isotype molecules, immune responses may also be raised against the primary ECGF-1 protein or against proteins or polypeptides created by translation of alternative reading frames.

At least 6 other SNP's have been found in hECGF-1 which may also give rise to immunological differences for the alleles in the context of different HLA isotypes and which, depending on the HLA presenting molecule, will constitute more polymorphic hECGF-1 minor histocompatibility antigens according to this invention.

TABLE 1
SNP's in the hECGF-1 cDNA sequence, Grey shaded boxes: non-synonymous SNP, in
brackets: amino acid encoded by codon (single letter code).

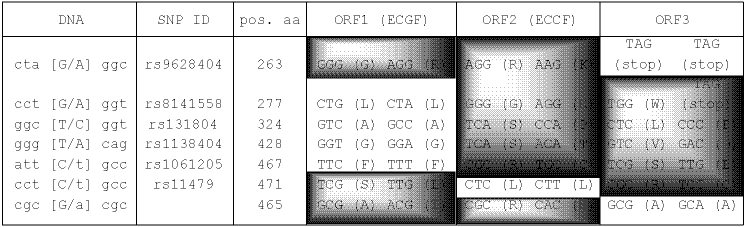

A polymorphic minor histocompatibility antigen in the hECGF-1 gene and its encoded polypeptides according to the invention may also reside in polymorphisms such as SNPs that are located in intronic sequences of the gene. Polymorphisms in intronic sequences may result in alteration of splice acceptor or donor sites, or by any other means result in altered splicing or coding differences in open reading frames that are not part of the hECGF-1 cDNA but are nevertheless to some extent translated in vivo. Any polymorphism comprised within the genomic sequence of the hECGF-1 gene, located on chromosome 22, such as, but not limited to the ones represented in table 2 may be suitably applied according to this invention to provide suitable ECGF-1 encoded mHags.

TABLE 2
SNP's in hECGF-1 intronic sequences

| DNA | SNP ID |
|---|---|
| CTT [C/T] CCT | rs131800 |
| gga [C/T] cac | rs131802 |
| ATT [A/G] ATC | rs470119 |
| GTA [A/G] ATG | rs7289374 |
| aca [A/C] ggc | rs131801 |
| ATC [C/G] AGA | rs762560 |
| aaa[ —/AAAA] GCT | rs131803 |
| TCT [A/C] CAA | rs3180095 |
| ctt [C/T] ctc | rs9628205 |
| GAT [C/G] CAG | rs762559 |

(Ref: http://bioinfo.weizmann.ac.il)

Furthermore, the mHag polymorphism may be a polymorphism or SNP or mutation that is causative (when homozygous or compound heterozygous) for the human genetic disorder MNGIE. Examples of MNGIE mutations discovered to date and resulting amino acid changes in all three reading frames are listed in table 3.

Whether an arbitrary SNP or any other polymorphism will yield a minor or a major histocompatibility antigen that can be presented in the context of a certain HLA molecule can be readily determined by the skilled artisan using computer modeling and applying bioinformatics tools, such as, but not limited to: HLA_BIND, SYFPEITHI, NetMHC and TEPITOPE 2000 analysis (37-42). Whether a certain fragment of the ECGF-1 protein or an alternative reading frame translation product will be degraded by the proteasome and displayed on HLA molecules can also be determined in vitro using commonly known techniques such as those described in 43, 44 and 45.

TABLE 3
MNGIE causing mutations in hECGF- 1, gray shaded boxes: non-synonymous codons, in
brackets: amino acid encoded by codon (single letter code).

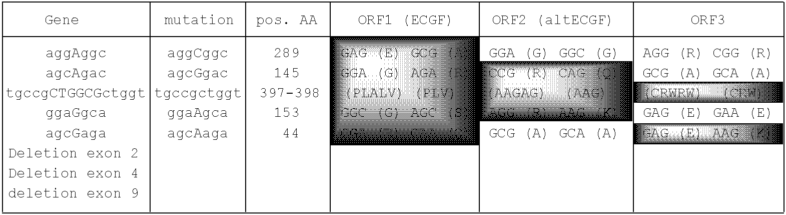

Hence, in one embodiment of the invention, this specification provides peptides comprising an amino acid sequence encoded by a reading frame as present in the nucleotide sequence of a transcript of a naturally occurring hECGF-1 allele, wherein the amino acid sequence comprises a polymorphic MHC class I or II minor histocompatibility binding peptide or fragment. A peptide of the invention is normally about 8 to 12 amino acids long, small enough for a direct fit in an HLA molecule, but it may also be larger and presented by HLA molecules only after cellular uptake and processing by the proteasome before presentation in the groove of an HLA molecule. The peptide may also be N- and/or C-terminally capped or modified to prevent degradation, increase stability or uptake. An mHag comprising peptide according to this invention preferably comprises the gene product of a single nucleotide polymorphism (SNP). The SNP may be comprised in the coding regions or exons of hECGF-1, or may be located in intronic sequences, affecting splicing or affecting cryptic messengers and translation products. The polymorphism may also be a MNGIE causing allele. In particular the polymorphism may be selected from the known polymorphisms in hECGF-1 selected from any of tables 1, 2 and 3, although newly discovered mutations and SNP's may also prove useful and result in a differential immune response between individuals. In a most preferred embodiment of the invention, the mHag to be used is a polymorphism affecting a change of amino acid 465 of hECGF-1, which gives rise to at least 4 peptide translation products, i.e. both alleles, from 2 out of the 3 different reading frames, containing an mHag as comprised in SEQ ID No's 1, 2, 3 or 4.

It is important to point out that for use according to this invention, whether a given polymorphism results in a polymorphic hECGF-1 encoded mHag and is useful for raising an immune response, graft vs. leukemia or graft vs. tumor, will depend on the genetic makeup and in particular HLA isotypes of both a donor and acceptor/recipient and their respective differences. Even if donor and recipient are identical with respect to their ECGF-1 alleles, but differ in their HLA isotypes an immune response may arise if T cells recognize a self antigen in the context of a different HLA allele (i.e. as a ‘non-self’-configuration) as foreign antigen. Application of such antigenic reactions against ECGF-1 is still within the scope of the current invention.

The peptides according to this invention may be comprised, used or applied in the context of an MHC class I or MHC class II molecule, for instance for raising or enhancing an T cell immune response, in order to select for binding or interacting T cell receptors or alternatively for antibodies capable of binding the peptides of the invention, optionally in the context of a certain HLA isotype molecule.

In another embodiment the invention provides nucleic acid molecules encoding the before mentioned peptides comprising ECGF-1 mHags. These nucleic acids may be useful as means for producing the peptides of the invention or alternatively as pharmaceutical compositions or DNA vaccines, to elicit, accelerate or enhance an immune response, in particular a desirable graft vs. tumor response. Preferably, the nucleic acids of the invention may be comprised in a nucleic acid vector, such as a plasmid, cosmid, an RNA or DNA phage or virus, or any other replicable nucleic acid molecule, and are most preferably operably linked to regulatory sequences such as (regulatable) promoters, initiators, terminators and/or enhancers.

In another embodiment, the current invention provides a T cell receptor capable of interacting with the ECGF-1 polymorphic mHags containing peptides and in particular nucleic acid molecules encoding such a T cell receptor, optionally comprised within a nucleic acid vector for expression and/or cloning purposes. T cell receptors and in particular nucleic acids encoding TCR's according to the invention may be applied to transfer a TCR from one T cell to another T cell and generate new T cell clones, for instance T cell clones that essentially are of the genetic make-up of a graft donor. The method to provide T cell clones capable of recognizing an mHag comprising peptide according to the invention may be generated and can be specifically targeted to tumor cells expressing a ECGF-1 polymorphic mHag in a graft or DLI recipient subject. Hence the invention provides T lymphocytes comprising a T cell receptor capable of interacting with a polymorphic mHag encoded by a reading frame in hECGF-1, which may be recombinant or naturally selected T lymphocytes. T lymphocytes of the invention may be used for the methods and pharmaceutical compositions of the invention. This specification thus provides at least two methods for producing a cytotoxic T lymphocyte of the invention, comprising the step of bringing undifferentiated lymphocytes into contact with a polymorphic hECGF-1 minor histocompatibility antigen under conditions conducive of triggering an immune response, which may be done in vitro or in vivo for instance in a patient receiving a graft. Alternatively, it may be carried out in vitro by cloning a gene encoding the TCR specific for a polymorphic hECGF-1 minor histocompatibility antigen, which may be obtained from a cell obtained from the previous method, into a host cell and/or a host lymphocyte obtained from a graft recipient, preferably a cytotoxic T lymphocyte (CTL).

In another embodiment, the invention provides new means, pharmaceuticals and/or medicaments, to treat malignancies expressing the ECGF-1 protein. The medicament is administered to a patient or subject suffering from a malignancy in an amount sufficient to at least reduce the growth of the malignancy, preferably reduce the malignancy in size and most preferably eradicates the malignancy. The patient or subject to be treated preferably is a human. The malignancies to be treated according to this invention may be any neoplastic disease expressing ECGF-1, comprising all hematological malignancies such as leukemia's, lymphoma's and (multiple) myeloma's, and all solid tumors, ranging from (benign) adenoma's and polyps to invasive and/or metastatic carcinoma's. Solid tumors which are dependent on angiogenesis and/or which have undergone infiltration with ECGF-1 expressing and/or producing macrophages, or solid tumors in which the angiogenesis is at least partly mediated by ECGF-1 from tumor cells or infiltrating macrophages, are particularly suitable for treatment according to this invention.

The methods and means of the invention are particularly suitable to be applied in the context of a subject that has undergone an allogeneic stem cell transplant, in for instance a hematopoietic stem cell transplant or donor lymphocyte infusion (DLI). The transplant is preferably, but not necessarily, HLA matched and comprises an allogeneic donor which does not comprise at least one hECGF-1 allele that is present in the recipient of the transplant or graft and therefore seen as ‘foreign’ or ‘non-self’ by the graft. Alternatively, donor and recipient may have identical HECGF-1 alleles and are HLA mismatched, whereby the HLA mismatch is capable of inducing an ECGF-1 specific graft vs. tumor response by presenting hECGF-1 peptides in a different HLA context, recognized by the graft derived T-cells as non-self. Genotyping of donor and recipient subjects for HLA or ECGF-1 alleles is a routine procedure that can be carried out by any skilled artisan using several standard, textbook techniques.

The peptides according to the invention, which as defined before comprise an ECGF-1 encoded polymorphic mHag, or lymphocytes carrying a T cell receptor capable of interacting with the peptides of the invention in the context of an HLA molecule, may be used for the manufacture of pharmaceutical compositions and medicaments for the treatment of a subjects suffering from malignancies expressing hECGF-1. The pharmaceutical compositions according to the invention will help to elicit, accelerate, enhance or prolong an effective immune response in the subject to be treated, in particular a desirable graft versus tumor T cell immune response. A graft vs. tumor response is in particular suitable for removal of minimal residual disease or metastases after chemotherapy of hematological cancers or after radiotherapy, chemotherapy or surgical resection in the case of operable solid tumors. A graft vs. tumor response is preferably a graft vs. hematological cancer response. A graft versus tumor response against solid tumors is preferably applied to those tumors in organs or tissues which are dispensable or replaceable, and which may be completely eradicated by the graft vs. host and/or graft vs. tumor response without serious adverse consequences. Such organs or tissues comprise testes, kidneys, ovaria, breastglands/tissues, prostate, thyroid, cervix, uterus and pancreas. In a particular embodiment, the method and the medicaments of the invention may be applied to inhibit tumor vascularization and/or to enhance apoptosis of tumor cells in response to anti-tumor treatment. The invention may be used as a primary method of treatment or as an adjuvant or follow-up treatment.

In a particular embodiment, the graft (stem) cells, in particular bone marrow/lymphocyte stem cells, may be primed prior to the transplant in the donor, by bringing them into contact with the ECGF-1 mHag's and/or medicaments according to the invention, in order to stimulate or accelerate an anti-tumor immune response against the ECGF-1 mHag displaying tumor cells, after transplantation of the graft to the recipient.

The medicaments and pharmaceutical compositions according to the invention may be formulated using generally known and pharmaceutically acceptable excipients customary in the art and for instance described in Remington, The Science and Practice of Pharmacy, 21^{nd} Edition, 2005, University of Sciences in Philadelphia. In particular immune modulating compounds and adjuvants may be suitably selected and applied by the skilled artisan, such as immune modulators described in Current Protocols in Immunology, Wiley Interscience 2004.

In yet another embodiment the invention provides for a human or humanized antibody, or a fragment thereof, specific for a polymorphic hECGF-1 minor histocompatibility antigen, the antigen optionally being in the context of an HLA molecule. Antibodies according to the invention may be used for therapeutic and pharmaceutical purposes and aiding in an anti-tumor immune response but may also be used for diagnostic purposes, in order to monitor tumors or tumor cells whether hECGF-1 mHag is displayed by these cells, or which polymorphic hECGF-1 mHags are displayed. An antibody according to the invention is preferably capable of binding to or interacting with polymorphic hECGF-1 peptides, optionally in the context of an HLA molecule. The antibody may also be an antibody raised in any other mammal, which may be humanized using conventional techniques. The antibody of the invention may be directly or indirectly labeled using conventional techniques. Suitable labels comprise fluorescent moieties (such as; GFP, FITC, TRITC, Rhodamine), enzymes (such as peroxidase, alkaline phosphatase), radioactive labels (³²P, ³⁵S, ¹²⁵I and others), immunogenic or other haptens or tags (biotin, digoxigenin, HA, 6His, LexA, Myc and others).

The antibodies and the peptides according to this invention may also be used to monitor graft anti-tumor responses by means of tetramer- or cytokine responses, such as the induction of interleukins and/or IFN-γ.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### FIGURE LEGENDS

FIG. 1. CTL clone RDR173 lyses tumor cells and hematopoietic cells. Lysis of tumor cells (a) patient-derived EBV-LCL cells (b) and donor-derived EBV-LCL cells (c) were tested in a CFSE-based cytotoxicity assay. Target cells were labeled with CFSE and incubated for 48 h with CTL clone RDR173 (E/T ratio 3:1). As a positive control, target cells were incubated with an HLA-A2-specific allo-reactive CTL clone (pos. CTL) and as negative control cells were incubated with a male-specific CTL clone (neg. CTL).

FIG. 2. CTL clone RDR173 recognizes a mHag presented in HLA-B7. (a)

Recognition of EBV-LCL cells were tested for recognition by CTL clone. (b) EBV-LCL expressing one or more HLA class I allele in common with the patient (A2, A3, B7, B60, Cw3) were incubated with CTL clone RDR173 for 24 h. Release of IFN-7 was determined using ELISA. HLA alleles that are shared with the patient are depicted in bold.

FIG. 3. CTL clone RDR173 recognizes an epitope translated in an alternative ORF. Deletion constructs derived from patient and donor ECGF-1 cDNA (a), constructs with the CTG codon at pos. 1441-1443 mutated into an AAG codon (a) and constructs with Kozak-ATG sequences in the primary or the alternative ORFs (b) were cloned into an expression vector and transfected into COS/HLA-B7 cells. Transfected cells were cocultered with CTL clone RDR173 for 24 h. Release of IFN-γ in the supernatant was measured by ELISA.

FIG. 4. Nucleotide sequence and protein translation of the partial ECGF 1 cDNA isolated from the cDNA library. The internal ATG codon of the primary ECGF-1 ORF and the CTG start codon of the alternative ORF are depicted in bold and underlined. The SNP and amino acid substitutions are indicated with arrows. The CTL epitope recognized by CTL clone RDR173 is boxed. Numbers are relative to the full-length ECGF cDNA and protein.

FIG. 5. CTL clone RDR173 recognizes the minimal epitope RPHAIRRPLAL. (a) Overlapping 9-, 10- and 11-mer peptides (1 μg/ml) were pulsed on donor-derived EBV-LCL cells for 2 h at 37° C. Peptide-pulsed EBV-LCLs were cocultured with CTL clone RDR173 for 24 h. Release of IFN-γ in the supernatant was measured by ELISA. (b) Increasing concentrations of the patient-derived peptide RPHAIRRPLAL (filled symbols) and the donor-derived peptide RPRAIRRPLAL (open symbols) were pulsed on donor-derived EBV-LCL cells for 2 h at 37° C. Peptide-pulsed EBV-LCLs were cocultured with CTL clone RDR173 for 24 h. Release of IFN-γ in the supernatant was measured by ELISA.

FIG. 6. CTL clone RDR173 lyses altECGF-1H-positive tumor cells. HLA-B7-expressing CML cells were labeled with CFSE and incubated with CTL clone RDR173. Melanoma cells were transduced with HLA-B7 and tested for recognition by CTL clone RDR173 in a CFSE-based cytotoxicity assay. As a control cells were transduced with retroviral construct containing only the marker gene. Target cells were also incubated with an HLA-B7-specific alloreactive T cell clone to demonstrate functional HLA-B7 expression

FIG. 7. Pulsing target cells with the 23-mer peptide leads to efficient processing and presentation LB-ECGF-1H epitope. CD 14+ cells were pulsed with titrated concentrations of the 11- and 23-mer hECGF-1 peptide for 4 h at 37° C. Target cells (3×10⁴) were incubated with CTL clone RDR173 (10⁴) for 24 h at 37° C. Release of IFN-γ in the supernatant was measured by ELISA.

FIG. 8. The LB-ECGF-1H epitope can be processed and presented by various target cells upon pulsing with the 23-mer peptide. HLA-B7-positive EBV-LCL cells (EBV), peripheral blood lymphocytes (PBL), phytohemagglutinin activated T cells (PHA blasts), CD14⁺ cells (CD14) and CD19⁺ cells (CD19) were pulsed with titrated concentrations of the 23-mer HECGF-1 peptide for 4 h at 37° C. Target cells (3×10⁴) were incubated with CTL clone RDR173 (10⁴) for 24 h at 37° C. Release of IFN-γ in the supernatant was measured by ELISA.

### EXAMPLES

### Isolation and Characterization of Tumor-Reactive T Cell Clone RDR173

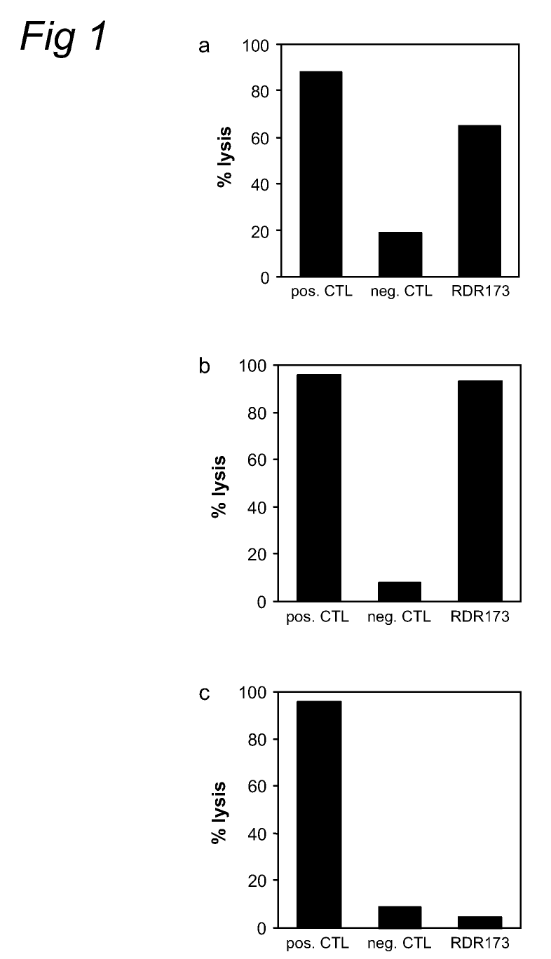

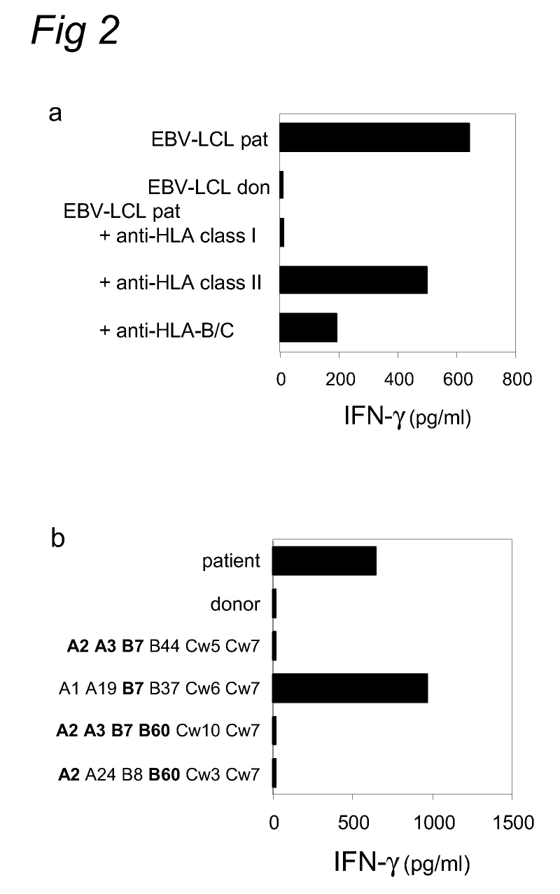

We isolated from peripheral blood of a patient successfully treated with DLI for relapsed MM, CD8+ CTL clones by direct cloning of T cells that produced IFN-γ upon stimulation with irradiated BM cells harvested from the patient before SCT (46). CTL clones recognizing several distinct antigens in the context of various HLA class I alleles were isolated including CTL clone RDR173. The tumor-reactivity of CTL clone RDR173 was demonstrated by the recognition of MM cells in the bone marrow from the moment of relapse using a CFSE-based cytotoxicity assay (47). To identify the tumor cells in the heterogeneous cell population, malignant cells were stained with PE-labeled anti-CD138 antibodies. As shown in FIG. 1a, malignant MM cells were lysed by CTL clone RDR173 similar to a control anti-HLA-A2-specific CTL clone, whereas they were not lysed by a male-specific, HLA-A1-restricted CTL clone (negative control CTL clone). In addition to tumor cells, CTL clone RDR173 also lysed the EBV-transformed lymphoblastoid cell line (EBV-LCL) cells (FIG. 1b), whereas donor-derived EBV-LCL cells were not recognized (FIG. 1c). Similar results were obtained for patient- and donor-derived PHA-blasts (data not shown). Recognition of patient-derived EBV-LCL cells could be blocked by antibodies against HLA class I and HLA-B/C alleles as shown in FIG. 2a. Screening a panel of EBV-LCL cells sharing HLA-B or HLA-C alleles with the patient showed that CTL clone RDR173 recognized an epitope in the context of HLA-B7 (FIG. 2b). To demonstrate the clonality of clone RDR173, the TCR α and β chains of CTL clone RDR137 were determined using TCR VB-specific monoclonal antibodies and TCR AV and BV family-specific oligonucleotides and sequencing. Since only one TCR α and one TCR β could be detected, the tumor-reactive CTL clone RDR173 was demonstrated to be a monoclonal CTL recognizing a mHag presented in HLA-B7.

### Identification of the Epitope Recognized by CD8+ CTL Clone RDR173

To identify the antigen that is recognized by CTL clone RDR173, cDNA expression cloning was performed based on the procedure as described by De Plaen et al. (48). Briefly, a cDNA library was constructed from EBV-LCL cells derived from the patient. Plasmid pools consisting of 40 cDNAs from the library were transfected into COS cells that were retrovirally transduced with HLA-B7 cDNA (COS/HLA-B7). Transfected COS/HLA-B7 cells were tested for recognition by CTL clone RDR173. One cDNA pool stimulated IFN-γ production of the CTL, and subcloning of this pool resulted in the isolation of a plasmid containing a 475-bp cDNA insert that was recognized by CTL clone RDR173 after transfection in COS/HLA-B7 cells. A BLAST search revealed that this cDNA insert was identical to the 3′ part (nt 1126-1600) of endothelial cell growth factor-1 (ECGF-1, also known as platelet-derived-ECGF or thymidine phosphorylase) cDNA. The incomplete cDNA fragment of ECGF-1 that we isolated from the cDNA library contained an internal ATG codon (nt 1169-1171) from which translation results in a protein identical to the C-terminal 137 amino acids of ECGF-1. Comparing the cDNA insert isolated from the cDNA library with donor-derived cDNA showed that these cDNA sequences were completely identical except for a G-to-A transition at nt 1526.

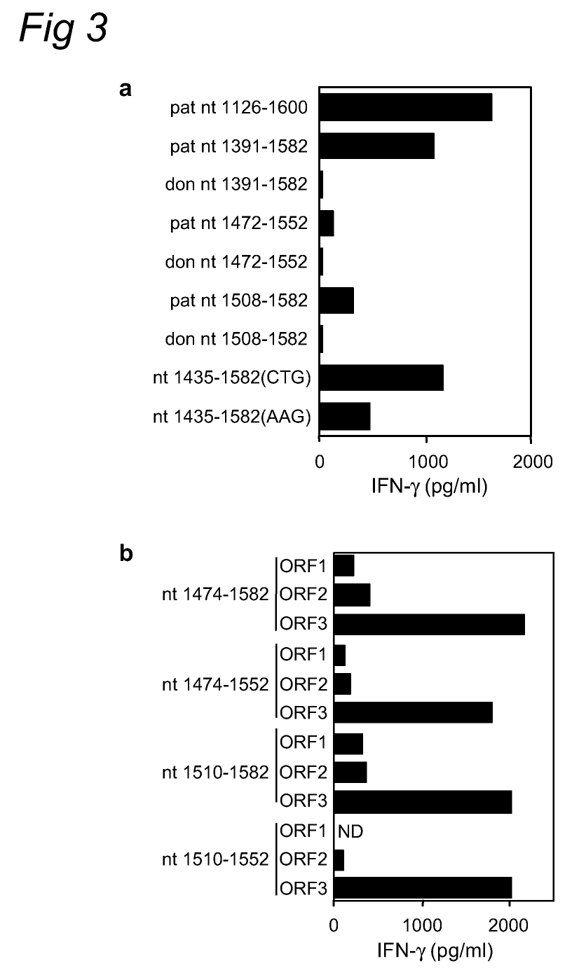

To determine whether this non-synonymous polymorphism encoded the epitope recognized by CTL clone RDR173, constructs were made containing nt 1391-1582, 1472-1552 and 1508-1582 of the cDNA insert, respectively, preceeded by Kozak-ATG sequences to ensure translation. The construct containing nt 1391-1582 was clearly recognized by CTL clone RDR173 upon transfection in COS/HLA-B7 (FIG. 3a). However, the smaller fragments containing nt 1472-1552 and 1508-1582 were hardly recognized. Even if the Ala-to-Thr substitution caused by the polymorphism is not present in the epitope itself, but affects antigen processing, e.g. proteasomal cleavage, at least one of these constructs was expected to be recognized by CTL clone RDR173. Donor-derived fragments of ECGF-1 were not recognized by CTL clone RDR173, confirming that the G-to-A transition at position 1526 was likely to be involved in the differential expression or recognition of the epitope.

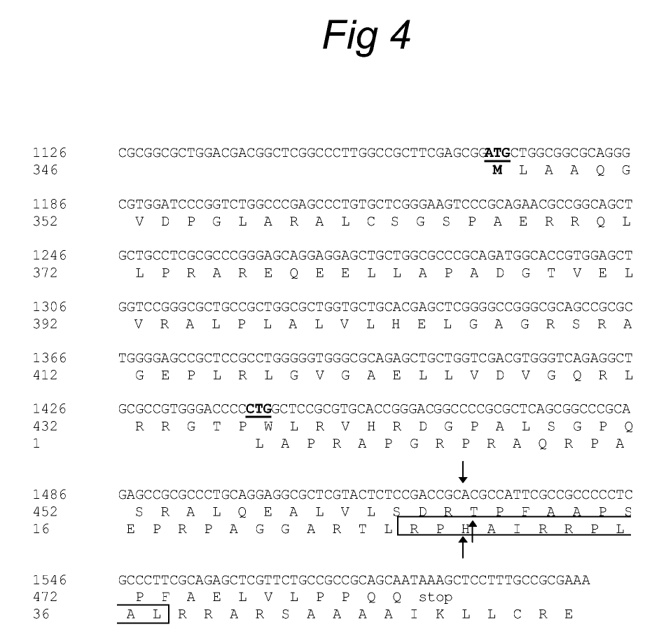

Analysis of nt 1391-1472 of the cDNA insert revealed the presence of a CTG codon (nt 1441-1443) capable of serving as an alternative start codon. Translation initiated by this CTG codon would result in a protein encoded by another ORF than ECGF-1 in which the SNP at nt 1526 caused an Arg-to-His substitution (FIG. 4). To investigate whether the epitope recognized by CTL clone RDR173 was encoded by the alternative ORF we made a construct in which the CTG codon was mutated to AAG. Transfection of COS/HLA-B7 cells with this constructs demonstrated decreased recognition (FIG. 3a), suggesting that translation starting at the CTG at nt 1441-1443 resulted in production of the epitope recognized by CTL clone RDR173.

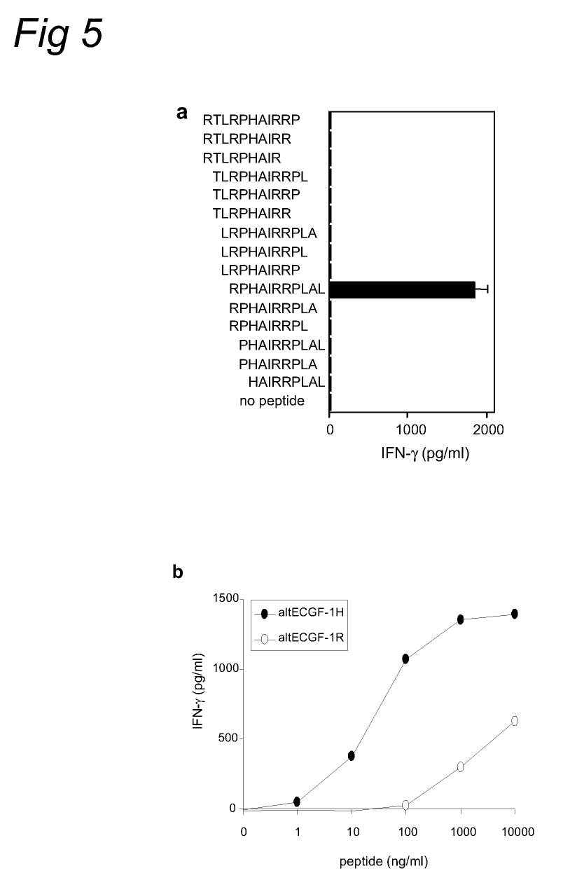

To further determine the location of the epitope recognized by CTL clone RDR173, smaller cDNA fragments with Kozak-ATG sequences in all three ORF were constructed and tested for recognition. Since the fragment containing nt 1510-1552 translated in the third ORF was still recognized by CTL clone RDR173 (FIG. 3b), the epitope was located within the 14 amino acid sequence encoded by this fragment. To identify the minimal peptide recognized by CTL clone RDR173, overlapping 9-, 10- and 11-mer peptides were synthesized, pulsed on donor-derived EBV-LCL cells and tested for recognition. FIG. 5a demonstrates that the 11-mer RPHAIRRPLAL peptide is the epitope recognized by CTL clone RDR173 (LB-ECGF-1H).

To reveal whether the differential recognition of recipient and donor cells by CTL clone RDR173 is due to differences in presentation of the epitope or recognition by the T cells, the donor-derived peptide RPRAIRPLAL (LB-ECGF-1R) was synthesized. EBV-LCL cells pulsed with the LB-ECGF-1R peptide were not recognized by CTL clone RDR173 (FIG. 5b). Since LB-ECGF-1H and LB-ECGF-1R bind to HLA-B7 with comparable affinities (EC50 of 14.2 and 26.2, respectively; data not shown), these data indicate that the amino acid substitution is critical for TCR recognition and affects a TCR contact residue.

### Analysis of LB-ECGF-1H-Specific Immune Response In Vivo

The magnitude of the LB-ECGF-1H-specific immune response in the peripheral blood of the MM patient responding to DLI was analyzed using tetrameric complexes. At the time of the clinical response, 5-7 weeks after DLI (49), 1.1% of the CD8+ T cells recognized the HLA-B7/LB-ECGF-1H tetramer (data not shown). Tetramer-positive T cells were cloned by FACSsorting and TCR α and β chains were determined from proliferating, HLA-B7/LB-ECGF-1H-positive clones. All clones expressed the same TCR α and β chain as clone RDR173 indicating that the LB-ECGF-1H-specific T cells in the peripheral blood of the patients were derived from a monoclonal T cell response.

### Population Frequency of LB-ECGF-1H/R Alleles

The disruption of a BssHII restriction site by the G-to-A transition at pos. 1526 was used to determine the population frequency of the LB-ECGF-1H and LB-ECGF-1R alleles. A 223-bp cDNA fragment was amplified from 70 unrelated individuals and digested with BssHII. PCR products obtained from the LB-ECGF-1H allele could not be cut by BssHII, whereas digestion of LB-ECGF-1R-derived PCR products resulted in two bands of 69 and 154 bp. Sixty-two persons were homozygous for the LB-ECGF-1R allele, whereas eight individuals were heterozygous for this polymorphism, indicating a frequency of 11% LB-ECGF-1H-positive individuals in the population. Individuals that were homozygous for the LB-ECGF-1H allele were not detected.

At least 6 other SNP's have been described in hECGF-1 which may also give rise to immunological differences (Table 1). We have analyzed the frequencies of these SNPs in a panel of unrelated individuals by sequencing. The frequencies of the alleles are shown in Table 4.

TABLE 4
Population frequencies of other SNP's in hECGF-1

| | pos. | Homozygous | | Homozygous | | |
|---|---|---|---|---|---|---|
| DNA | aa | wild type | Heterozygous | SNP | % SNP-pos | allele freq. |
| cta [G/C]ggc | 263 | 13/13 | 0/13 | 0/13 | 0 | |
| cct [G/A] ggt | 277 | 13/13 | 0/13 | 0/13 | 0 | |
| ggc [T/C] cag | 324 | 9/37 | 18/37 | 10/37 | 76 | 0.51 |
| ggg [T/A] cag | 428 | 32/37 | 5/37 | 0/37 | 14 | 0.07 |
| att [C/T] gcc | 467 | 14/14 | 0/14 | 0/14 | 0 | |
| cct [C/T] gcc | 471 | 25/37 | 10/37 | 2/37 | 32 | 0.19 |

### Expression of ECGF-1 in Normal Tissue and Malignancies

Analysis of the expression pattern of ECGF-1 in a microarray study of gene expression across tissues (http://symatlas.gnf.org/SymAtlas/; ref (50)) showed high expression of ECGF-1 in several cells of the hematopoietic system, in particular CD14+ monocytes and DC. Furthermore, the ECGF-1 gene was reported to be present in cDNA derived from lung, liver and heart. To confirm these data, we performed quantitative realtime RT-PCR for ECGF-1. In accordance with the DNA microarray study, we detected the highest levels of ECGF-1 expression in CD14+ cells (data not shown). However, we also detected high ECGF-1 expression in other PBMC, like CD4+, CD8+ and CD19+ cells. Furthermore, ECGF-1 was detected in lung, spleen, liver, thymus and heart. In all the other tissues, the ECGF-1 gene expression was low or absent.

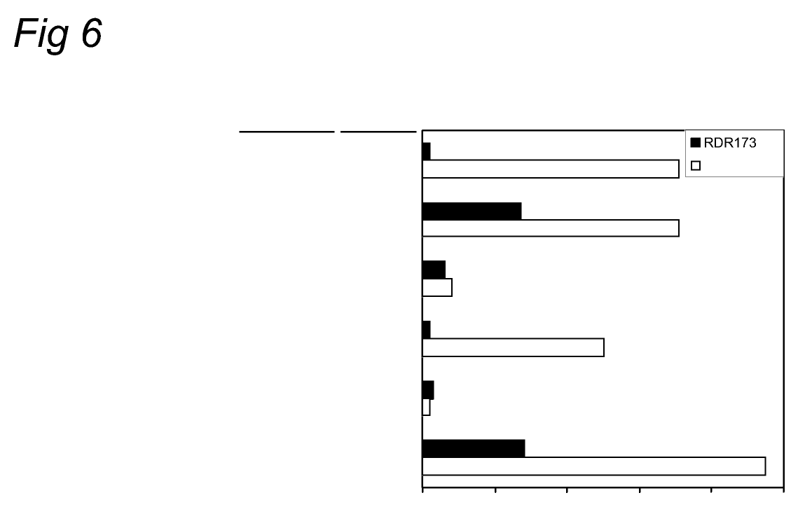

In hematological malignancies like multiple myeloma, CML, AML and ALL and in cell lines derived from solid tumors like melanoma, breast carcinoma, coloncarcinoma and ovarian carcinoma variable levels of expression of the ECGF-1 could be detected (data not shown). To investigate whether tumor cells other than MM cells could also be recognized by CTL clone RDR173, CML cells from a HLA-B7-expressing, LB-ECGF-1H-positive patient were tested for recognition by CTL clone RDR173. Furthermore, LB-ECGF-1H-positive melanoma cells in which low expression of the ECGF-1 gene could be detected were tested for recognition by CTL clone RDR173. Since the melanoma is HLA-B7-negative, these cells were retrovirally transduced with HLA-B7. As shown in FIG. 6, HLA-B7-expressing, LB-ECGF-1H-positive CML and melanoma cells were lysed by CTL clone RDR173, whereas HLA-B7-expressing, altECGF-1R-positive cells were not lysed by CTL clone RDR173. Similar results were obtained when release of IFN-γ was determined (data not shown). These data demonstrate that the LB-ECGF-1H-specific CTL clone RDR173 can be reactive against both hematological malignancies and solid tumors.

Processing and Presentation of the LB-ECGF-1H Epitope from Synthetic Peptides

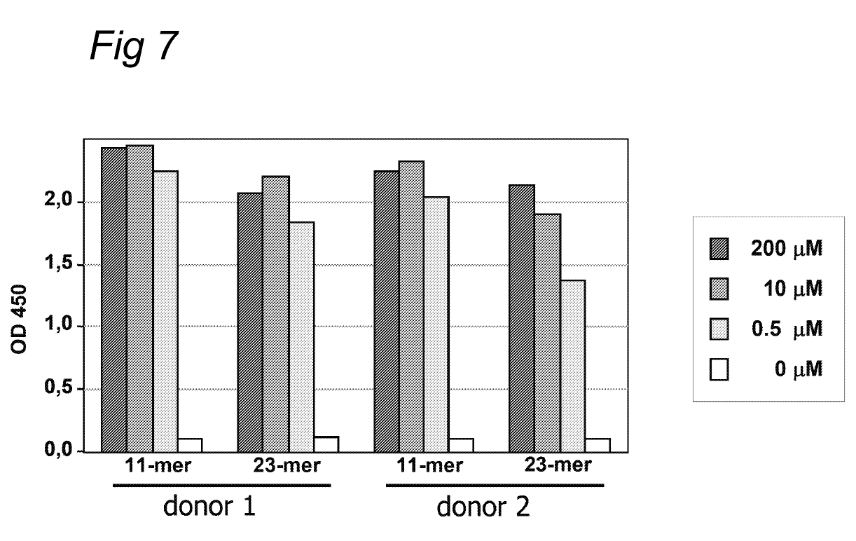

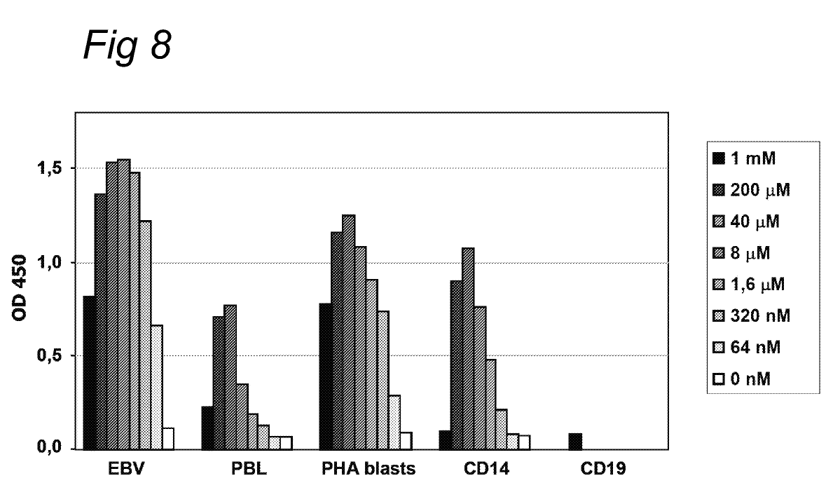

Furthermore, we have tested whether LB-ECGF-1H could also be processed and presented when long peptides were pulsed on target cells, indicating that the LB-ECGF-1H epitope will be presented in vivo when long peptides are used for vaccination. The 23-mer peptide RPAGGARTLRPHAIRRPLALRRA containing the epitope (depicted in bold) was synthesized. Target cells were pulsed with titrated concentration of the long peptide for 4 hours at 37° C. and tested for recognition by CTL clone RDR173. FIG. 7 demonstrates that pulsing target cells with the 23-mer peptide results in an efficient recognition by CTL clone RDR173, comparable to pulsing target cells with the 11-mer epitope. FIG. 8 shows that various target cells like EBV-LCL, PHA blast and CD14+ cells, have the ability to process and present the epitope upon pulsing with the 23-mer peptide.

### Materials and Methods

### Cell Culture

The mHAg-specific HLA-B7-restricted CD8+ CTL clone RDR173 was isolated from peripheral blood of a patient with relapsed MM responding to DLI after HLA-matched allogeneic SCT using the IFN-γ-secretion assay (46). CTL clone RDR173 was cultured by stimulation with irradiated (50 Gy) allogeneic PBMC and patient-derived EBV-transformed B cells in IMDM (Cambrex, Verviers, Belgium) supplemented with 5% human AB0 serum, 5% fetal bovine serum (FBS), 100 U/ml IL-2 (Chiron, Amsterdam, the Netherlands) and 0.8 μg/ml phytohaemagglutinin (PHA, Murex Biotec Limited, Dartford, United Kingdom). EBV-transformed lymphoblastoid cell lines (EBV-LCL), COS cells and melanoma cell lines were maintained in IMDM (Cambrex) containing 8% FBS.

### CFSE-Based Cytotoxicity Assay

To determine lysis by CTL clone RDR173 of malignant cells in a heterogenous population of target cells, a CFSE-based cytotoxicity assay was performed as described before (47). Briefly, PBMC, bone marrow cells or tumor cells were labeled with 2.5 μM CFSE and incubated with T cell clone RDR173 (E:T ratio 1:3) at 37° C. After 24 or 48 h, MM and CML cells were stained with PE-labeled anti-CD138 and anti-CD34 antibodies, respectively. Melanoma cells retrovirally transduced with HLA-B7 were stained with APC-labeled antibodies against the marker gene NGFR. To allow quantitative analysis, 104 Flow-Count Fluorospheres (Coulter Corporation, Miami, USA) were added, and samples were directly analyzed by flowcytometry.

### IFN-γELISA

EBV-LCL cells were seeded at 3×104 cells/well together with 5×103 T cells in 96-well round bottom plate. For blocking studies, target cells were preincubated with antibodies against HLA class I (w6/32), HLA class II (PdV5.2) or HLA-B/C alleles (B1.23.2) for 30 min at room temperature. After 24 h incubation at 37° C., release of IFN-γ in the supernatant was measured using standard ELISA (Sanguin, Amsterdam, the Netherlands).

### Analysis of TCRVα and TCRβ Chains

TCR AV and BV gene usage of the CTL clones was determined by PCR and sequencing as described before (51).

Construction of cDNA Library

A cDNA library was constructed using the SuperScript Choice System kit (Invitrogen, Breda, the Netherlands). RNA was isolated from the patient-derived EBV-LCL using Trizol (Invitrogen) according to the manufacturer's instructions and further purified using the RNA Cleanup protocol of the RNeasy kit (QIAGEN, Venlo, the Netherlands). Poly (A)+MRNA was isolated using the PolyATract mRNA Isolation System (Promega, Leiden, the Netherlands), and converted to cDNA using an oligo-d(T) primer containing a NotI restriction site at the 5′ end. After double strand cDNA synthesis, the cDNA was ligated to BamHI-EcoRI adapters (Stratagene, La Jolla, Calif., USA), digested with NotI, and size-fractionated using column chromatography. Fractions containing the largest cDNA fragments were ligated into BamHI and NotI sites of the pCR3.1 expression vector (Invitrogen). Ligation products were transformed into E. Coli Top10 bacteria, and transformed clones were selected with ampicillin. The library was divided into pools of ˜40 cDNA clones. Each pools was amplified in liquid culture for 4 h, and plasmid DNA was isolated using QIAprep 96 Turbo Miniprep Kit (QIAGEN).

### Transfection of COS Cells and CTL Stimulation Assay

To identify the gene encoding the epitope that is recognized by CTL clone RDR173, COS cells were transfected with the cDNA library, and tested for recognition. Briefly, COS cells retrovirally transduced with the HLA-B7 cDNA were plated in flat-bottom 96-well plate at 2×104 cells/well, and incubated overnight at 37° C. Approximately 80 ng of plasmid DNA from each pool was mixed with 0.8 μl lipofectamine (Invitrogen) in 50 μl OptiMEM medium (Invitrogen). Transfection mixtures were incubated for 30 min at room temperature, and added to the COS/HLA-B7 cells. After 5 h of incubation at 37° C., 50 μl IMDM/10% FBS was added. Twenty-four hours after transfection, 3×103 CD8+CTL were added to transfected COS cells. After 24 h of coculture at 37° C., the release of IFN-7 in the supernatant was determined using standard ELISA (Sanguin, Amsterdam, the Netherlands).

### Tetrameric HLA Class I/Peptide Complexes and Flow Cytometric Analysis

PE-conjugated tetrameric complexes of HLA-B7 molecules with the ECGF-1-derived peptide RPHAIRRPLAL were constructed as previously described (52) with minor modifications. For cytometric analysis and FACSort experiments, cells were labeled with tetrameric complexes for 2 h at 4° C. in RPMI without phenol, supplemented with 2% FBS. Cells were counterstained with FITC-labeled antibodies against CD4 (Caltag) and CD40 (Serotech) or CD8 (Caltag).

### Screening for Population Frequency

RNA was isolated from PBMC using Trizol (Invitrogen) according to the manufacturer's instructions and transcribed to cDNA using M-MLV reverse transcriptase (Invitrogen). PCR was performed in 50 μl reaction mixture containing 1× buffer, 300 nM forward primer (5′-TATAAGATCTGCCACCATGGGCGCAGAGC-TGCTGGTC-3′), 300 nM reverse primer (5′-TACGCGGCCGCTTATTGCTGCGGC-GGCAGAAC-3′), 500 μM dNTP, GC-rich solution (Roche, Indianapolis, USA), 1 U PWO Superyield DNA polymerase (Roche). Amplification using the following PCR program 2′ 95° C., 30 cycli of 15″ 95° C., 30″ 55° C., 1′ 72° C., and final elongation for 7′ at 72° C. resulted in a fragment of 223 bp that was digested with 10 U BssHII for 2 h at 50° C. and analyzed on a 2% agarose gel. Digestion of PCR product derived from the LB-ECGF-1R-B7 allele results in bands of 69 and 154 bp, whereas amplification and digestion of the LB-ECGF-1H-B7 allele results in a single band of 223 bp.

### Quantitative RT-PCR

Quantitative real-time PCR was performed for the commercially available Human Blood fraction MTC Panel, Human MTC Panel I and II (BD Biosciences, Alphen aan de Rijn, the Netherlands). Using Primer Express software (Applied Biosystems, Foster City, Calif., USA), forward primer 5′-CGGAATCCTATATG-CAGCCAG-3′, reverse primer 5′-CCCTCCGAACTTAACGTCCA-3′ and probe 5′-(TET)-TGCCACTCATCACAGCCTCCATTCTC-(TAMRA)-3′ were designed for ECGF-1. PCR was performed in 50 μl reaction mixture containing 1× Taqman buffer, 300 nM of each primer, 120 nM probe, 200 μM dNTP, 3 mM MgCl2, 1.25 U AmpliTaq Gold polymerase and 10-100 ng of cDNA sample. PCR amplification was also performed for the housekeeping gene porphobilogen deaminase (PBGD) using forward primer 5′-GGCAATGCGGC-TGCAA-3′, reverse primer 5′-GGGTACCCACGCGAATCAC-3′ and probe 5′(TET)-CTCATCTTTGGGCTGTTTTCTTCCGCC-(TAMRA)-3′. For PBGD, PCR was performed in 50 μl reaction mixture containing 1× Taqman-buffer, 300 nM of each primer, 120 nM probe, 200 μM dNTP, 4 mM MgCl2, 1.25 U AmpliTaq Gold polymerase and 10-100 ng of cDNA sample. Amplification was started with 10′ 95° C., followed by 55 cycli of 15″ 95° C., 30″ 60° C. and 30″ 60° C. Expression of ECGF-1 was normalized to expression of the housekeeping gene.

### REFERENCES

1. Rosenberg, S. A., J. C. Yang, and N. P. Restifo. 2004. Cancer immunotherapy: moving beyond current vaccines. Nat. Med. 10:909-915.
2. Mocellin, S., S. Mandruzzato, V. Bronte, M. Lise, and D. Nitti. 2004. Part I: Vaccines for solid tumours. Lancet Oncol. 5:681-689.
3. Kolb, H. J., A. Schattenberg, J. M. Goldman, B. Hertenstein, N. Jacobsen, W. Arcese, P. Ljungman, A. Ferrant, L. Verdonck, D. Niederwieser, F. van Rhee, J. Mittermueller, T. de Witte, E. Holler, and H. Ansari. 1995. Graft-versus-leukemia effect of donor lymphocyte transfusions in marrow grafted patients. Blood 86:2041-2050.
4. Kolb, H. J., J. Mittermuller, C. Clemm, E. Holler, G. Ledderose, G. Brehm, M. Heim, and W. Wilmanns. 1990. Donor leukocyte transfusions for treatment of recurrent chronic myelogenous leukemia in marrow transplant patients. Blood 76:2462-2465.
5. Collins, R. H., Jr., O. Shpilberg, W. R. Drobyski, D. L. Porter, S. Giralt, R. Champlin, S. A. Goodman, S. N. Wolff, W. Hu, C. Verfaillie, A. List, W. Dalton, N. Ognoskie, A. Chetrit, J. H. Antin, and J. Nemunaitis. 1997. Donor leukocyte infusions in 140 patients with relapsed malignancy after allogeneic bone marrow transplantation. J. Clin. Oncol. 15:433-444.
6. Falkenburg, J. H., van de Corp, E. W. Marijt, and R. Willemze. 2003. Minor histocompatibility antigens in human stem cell transplantation. Exp. Hematol. 31:743-751.
7. Molldrem, J. J., E. Clave, Y. Z. Jiang, D. Mavroudis, A. Raptis, N. Hensel, V. Agarwala, and A. J. Barrett. 1997. Cytotoxic T lymphocytes specific for a nonpolymorphic proteinase 3 peptide preferentially inhibit chronic myeloid leukemia colony-forming units. Blood 90:2529-2534.
8. den Haan, J. M. M., L. M. Meadows, W. Wang, J. Pool, E. Blokland, T. L. Bishop, C. Reinhardus, J. Shabanowitz, R. Offringa, D. F. Hunt, V. H. Engelhard, and E. Goulmy. 1998. The minor histocompatibility antigen HA-1: A diallelic gene with a single amino acid polymorphism. Science 279:1054-1057.
9. Denhaan, J. M. M., N. E. Sherman, E. Blokland, E. Huczko, F. Koning, J. W. Drijfhout, J. Skipper, J. Shabanowitz, D. F. Hunt, V. H. Engelhard, and E. Goulmy. 1995. Identification of A Graft-Versus-Host Disease-Associated Human Minor Histocompatibility Antigen. Science 268:1476-1480.
10. Pierce, R. A., E. D. Field, T. Mutis, T. N. Golovina, C. Kap-Herr, M. Wilke, J. Pool, J. Shabanowitz, M. J. Pettenati, L. C. Eisenlohr, D. F. Hunt, E. Goulmy, and V. H. Engelhard. 2001. The HA-2 minor histocompatibility antigen is derived from a diallelic gene encoding a novel human class I myosin protein. J. Immunol. 167:3223-3230.
11. Akatsuka, Y., T. Nishida, E. Kondo, M. Miyazaki, H. Taji, H. Tida, K. Tsujimura, M. Yazaki, T. Naoe, Y. Morishima, Y. Kodera, K. Kuzushima, and T. Takahashi. 2003. Identification of a polymorphic gene, BCL2A1, encoding two novel hematopoietic lineage-specific minor histocompatibility antigens. Journal of Experimental Medicine 197:1489-1500.
12. Meadows, L., W. Wang, J. M. den Haan, E. Blokland, C. Reinhardus, J. W. Drijfhout, J. Shabanowitz, R. Pierce, A. I. Agulnik, C. E. Bishop, D. F. Hunt, E. Goulmy, and V. H. Engelhard. 1997. The HLA-A*0201-restricted H-Y antigen contains a posttranslationally modified cysteine that significantly affects T cell recognition. Immunity 6:273-281.
13. Pierce, R. A., E. D. Field, J. M. den Haan, J. A. Caldwell, F. M. White, J. A. Marto, W. Wang, L. M. Frost, E. Blokland, C. Reinhardus, J. Shabanowitz, D. F. Hunt, E. Goulmy, and V. H. Engelhard. 1999. Cutting edge: the HLA-A*0101-restricted HY minor histocompatibility antigen originates from DFFRY and contains a cysteinylated cysteine residue as identified by a novel mass spectrometric technique. J. Immunol. 163:6360-6364.
14. Torikai, H., Y. Akatsuka, M. Miyazaki, E. H. Warren, III, T. Oba, K. Tsujimura, K. Motoyoshi, Y. Morishima, Y. Kodera, K. Kuzushima, and T. Takahashi. 2004. A novel HLA-A*3303-restricted minor histocompatibility antigen encoded by an unconventional open reading frame of human TMSB4Y gene. J. Immunol. 173:7046-7054.
15. Vogt, M. H., E. Goulmy, F. M. Kloosterboer, E. Blokland, R. A. de Paus, R. Willemze, and J. H. Falkenburg. 2000. UTY gene codes for an HLA-B60-restricted human male-specific minor histocompatibility antigen involved in stem cell graft rejection: characterization of the critical polymorphic amino acid residues for T-cell recognition. Blood 96:3126-3132.
16. Vogt, M. H., J. W. van den Muijsenberg, E. Goulmy, E. Spierings, P. Kluck, M. G. Kester, R. A. van Soest, J. W. Drijfhout, R. Willemze, and J. H. Falkenburg. 2002. The DBY gene codes for an HLA-DQ5-restricted human male-specific minor histocompatibility antigen involved in graft-versus-host disease. Blood 99:3027-3032.
17. Warren, E. H., M. A. Gavin, E. Simpson, P. Chandler, D. C. Page, C. Disteche, K. A. Stankey, P. D. Greenberg, and S. R. Riddell. 2000. The human UTY gene encodes a novel HLA-B8-restricted H-Y antigen. J. Immunol. 164:2807-2814.
18. Wang, W., L. R. Meadows, J. M. den Haan, N. E. Sherman, Y. Chen, E. Blokland, J. Shabanowitz, A. I. Agulnik, R. C. Hendrickson, C. E. Bishop, and 1995. Human H-Y: a male-specific histocompatibility antigen derived from the SMCY protein. Science 269:1588-1590.
19. Randolph, S. S., T. A. Gooley, E. H. Warren, F. R. Appelbaum, and S. R. Riddell. 2004. Female donors contribute to a selective graft-versus-leukemia effect in male recipients of HLA-matched, related hematopoietic stem cell transplants. Blood 103:347-352.
20. Spierings, E., A. G. Brickner, J. A. Caldwell, S. Zegveld, N. Tatsis, E. Blokland, J. Pool, R. A. Pierce, S. Mollah, J. Shabanowitz, L. C. Eisenlohr, P. van Veelen, F. Ossendorp, D. F. Hunt, E. Goulmy, and V. H. Engelhard. 2003. The minor histocompatibility antigen HA-3 arises from differential proteasome-mediated cleavage of the lymphoid blast crisis (Lbc) oncoprotein. Blood 102:621-629.
21. Brickner, A. G., E. H. Warren, J. A. Caldwell, Y. Akatsuka, T. N. Golovina, A. L. Zarling, J. Shabanowitz, L. C. Eisenlohr, D. F. Hunt, V. H. Engelhard, and S. R. Riddell. 2001. The immunogenicity of a new human minor histocompatibility antigen results from differential antigen processing. Journal of Experimental Medicine 193:195-205.
22. Murata, M., E. H. Warren, and S. R. Riddell. 2003. A human minor histocompatibility antigen resulting from differential expression due to a gene deletion. Journal of Experimental Medicine 197:1279-1289.
23. Dolstra, H., H. Fredrix, F. Maas, P. G. Coulie, F. Brasseur, E. Mensink, G. J. Adema, T. M. de Witte, C. G. Figdor, and van de Wiel-van Kemenade. 1999. A human minor histocompatibility antigen specific for B cell acute lymphoblastic leukemia. Journal of Experimental Medicine 189:301-308.
24. Toi, M., S. Hoshina, T. Taniguchi, Y. Yamamoto, H. Ishitsuka, and T. Tominaga. 1995. Expression of platelet-derived endothelial cell growth factor/thymidine phosphorylase in human breast cancer. Int. J. Cancer 64:79-82.
25. Imazano, Y., Y. Takebayashi, K. Nishiyama, S. Akiba, K. Miyadera, Y. Yamada, S. Akiyama, and Y. Ohi. 1997. Correlation between thymidine phosphorylase expression and prognosis in human renal cell carcinoma. J. Clin. Oncol. 15:2570-2578.
26. Reynolds, K., F. Farzaneh, W. P. Collins, S. Campbell, T. H. Bourne, F. Lawton, A. Moghaddam, A. L. Harris, and R. Bicknell. 1994. Association of ovarian malignancy with expression of platelet-derived endothelial cell growth factor. J. Natl. Cancer Inst. 86:1234-1238.
27. Fujiwaki, R., K. Hata, K. Tida, Y. Maede, M. Koike, and K. Miyazaki. 1999. Thymidine phosphorylase expression in progression of cervical cancer: correlation with microvessel count, proliferating cell nuclear antigen, and apoptosis. J. Clin. Pathol. 52:598-603.
28. Tokunaga, Y., H. Hosogi, T. Hoppou, M. Nakagami, A. Tokuka, and K. Ohsumi. 2002. Prognostic value of thymidine phosphorylase/platelet-derived endothelial cell growth factor in advanced colorectal cancer after surgery: evaluation with a new monoclonal antibody. Surgery 131:541-547.
29. Ikeda, R., T. Furukawa, M. Kitazono, K. Ishitsuka, H. Okumura, A. Tani, T. Sumizawa, M. Haraguchi, M. Komatsu, H. Uchimiya, X. Q. Ren, T. Motoya, K. Yamada, and S. Akiyama. 2002. Molecular basis for the inhibition of hypoxia-induced apoptosis by 2-deoxy-D-ribose. Biochem. Biophys. Res. Commun. 291:806-812.
30. Kitazono, M., Y. Takebayashi, K. Ishitsuka, S. Takao, A. Tani, T. Furukawa, K. Miyadera, Y. Yamada, T. Aikou, and S. Akiyama. 1998. Prevention of hypoxia-induced apoptosis by the angiogenic factor thymidine phosphorylase. Biochem. Biophys. Res. Commun. 253:797-803.
31. Mori, S., S. Takao, R. Ikeda, H. Noma, Y. Mataki, X. Wang, S. Akiyama, and T. Aikou. 2002. Thymidine phosphorylase suppresses Fas-induced apoptotic signal transduction independent of its enzymatic activity. Biochem. Biophys. Res. Commun. 295:300-305.
32. Mori, S., S. Takao, R. Ikeda, H. Noma, Y. Mataki, X. Wang, S. Akiyama, and T. Aiko. 2001. Role of thymidine phosphorylase in Fas-induced apoptosis. Hum. Cell 14:323-330.
33. Ikeda, R., T. Furukawa, R. Mitsuo, T. Noguchi, M. Kitazono, H. Okumura, T. Sumizawa, M. Haraguchi, X. F. Che, H. Uchimiya, Y. Nakajima, X. Q. Ren, S. Oiso, I. Inoue, K. Yamada, and S. Akiyama. 2003. Thymidine phosphorylase inhibits apoptosis induced by cisplatin. Biochem. Biophys. Res. Commun. 301:358-363.
34. Hotchkiss, K. A., A. W. Ashton, R. S. Klein, M. L. Lenzi, G. H. Zhu, and E. L. Schwartz. 2003. Mechanisms by which tumor cells and monocytes expressing the angiogenic factor thymidine phosphorylase mediate human endothelial cell migration. Cancer Res. 63:527-533.
35. Nishino, I., A. Spinazzola, and M. Hirano. 1999. Thymidine phosphorylase gene mutations in MNGIE, a human mitochondrial disorder. Science 283:689-692.
36. Fox, S. B., A. Moghaddam, M. Westwood, H. Turley, R. Bicknell, K. C. Gatter, and A. L. Harris. 1995. Platelet-derived endothelial cell growth factor/thymidine phosphorylase expression in normal tissues: an immunohistochemical study. J. Pathol. 176:183-190.
37. Parker, K. C., M. A. Bednarek, and J. E. Coligan. 1994. Scheme for ranking potential HLA-A2 binding peptides based on independent binding of individual peptide side-chains. J. Immunol. 152:163-175. HLA_BIND (http://bimas.cit.nih.gov/molbio/hla_bind/)
38. Rammensee, H. G., T. Friede, and S. Stevanoviic. 1995. MHC ligands and peptide motifs: first listing. Immunogenetics 41:178-228. SYFPEITHI (http://www.syfpeithi.de/) and Rammensee, Bachmann, Stevanovic:MHC ligands and peptide motifs. Landes Bioscience 1997 (International distributor—except North America Springer Verlag GmbH & Co. KG, Tiergartenstr. 17, D-69121 Heidelberg
39. Buus, S., S. L. Lauemoller, P. Worning, C. Kesmir, T. Frimurer, S. Corbet, A. Fomsgaard, J. Hilden, A. Holm, and S. Brunak. 2003. Sensitive quantitative predictions of peptide-MHC binding by a ‘Query by Committee’ artificial neural network approach. Tissue Antigens 62:378-384.; NetMHC (http://www.cbs.dtu.dk/services/NetMHC/)
40. Nielsen, M., C. Lundegaard, P. Worning, S. L. Lauemoller, K. Lamberth, S. Buus, S. Brunak, and O. Lund. 2003. Reliable prediction of T-cell epitopes using neural networks with novel sequence representations. Protein Sci. 12:1007-1017.
41. Nielsen, M., C. Lundegaard, P. Worning, C. S. Hvid, K. Lamberth, S. Buus, S. Brunak, and O. Lund. 2004. Improved prediction of MHC class I and class II epitopes using a novel Gibbs sampling approach. Bioinformatics. 20:1388-1397.
42. Sturniolo, T., E. Bono, J. Ding, L. Raddrizzani, O. Tuereci, U. Sahin, M. Braxenthaler, F. Gallazzi, M. P. Protti, F. Sinigaglia, and J. Hammer. 1999. Generation of tissue-specific and promiscuous HLA ligand databases using DNA microarrays and virtual HLA class II matrices. Nat. Biotechnol. 17:555-561.; TEPITOPE (http://www.vaccinome.com/pages/597444/)
43. Tan, T. L., A. Geluk, M. Toebes, T. H. Ottenhoff, and J. W. Drijfhout. 1997. A novel, highly efficient peptide-HLA class I binding assay using unfolded heavy chain molecules: identification of HIV-1 derived peptides that bind to HLA-A*0201 and HLA-A*0301. J. Immunol. Methods 205:201-209.
44. Ottenhoff, T. H., A. Geluk, M. Toebes, W. E. Benckhuijsen, K. E. van Meijgaarden, and J. W. Drijfhout. 1997. A sensitive fluorometric assay for quantitatively measuring specific peptide binding to HLA class I and class II molecules. J. Immunol. Methods 200:89-97.
45. Toes, R. E., A. K. Nussbaum, S. Degermann, M. Schirle, N. P. Emmerich, M. Kraft, C. Laplace, A. Zwinderman, T. P. Dick, J. Muller, B. Schonfisch, C. Schmid, H. J. Fehling, S. Stevanovic, H. G. Rammensee, and H. Schild. 2001. Discrete cleavage motifs of constitutive and immunoproteasomes revealed by quantitative analysis of cleavage products. J. Exp. Med. 194:1-12.
46. Kloosterboer, F. M., S. A. Luxemburg-Heijs, R. A. van Soest, H. M. van Egmond, A. M. Barbui, M. P. Strijbosch, R. Willemze, and J. H. Falkenburg. 2005. Minor histocompatibility antigen-specific T cells with multiple distinct specificities can be isolated by direct cloning of IFNgamma-secreting T cells from patients with relapsed leukemia responding to donor lymphocyte infusion. Leukemia 19:83-90.
47. Jedema, I., N. M. van der Werff, R. M. Barge, R. Willemze, and J. H. Falkenburg. 2004. New CFSE-based assay to determine susceptibility to lysis by cytotoxic T cells of leukemic precursor cells within a heterogeneous target cell population. Blood 103:2677-2682.
48. De Plaen, E., C. Lurquin, B. Lethe, B. P. van der, V. Brichard, J. C. Renauld, P. Coulie, A. Van Pel, and T. Boon. 1997. Identification of genes coding for tumor antigens recognized by cytolytic T lymphocytes. Methods 12:125-142.
49. Marijt, W. A., M. H. Heemskerk, F. M. Kloosterboer, E. Goulmy, M. G. Kester, M. A. van der Hoom, S. A. Luxemburg-Heys, M. Hoogeboom, T. Mutis, J. W. Drijfhout, J. J. van Rood, R. Willemze, and J. H. Falkenburg. 2003. Hematopoiesis-restricted minor histocompatibility antigens HA-1- or HA-2-specific T cells can induce complete remissions of relapsed leukemia. Proc. Natl. Acad. Sci. U.S.A 100:2742-2747.
50. Su, A. I., M. P. Cooke, K. A. Ching, Y. Hakak, J. R. Walker, T. Wiltshire, A. P. Orth, R. G. Vega, L. M. Sapinoso, A. Moqrich, A. Patapoutian, G. M. Hampton, P. G. Schultz, and J. B. Hogenesch. 2002. Large-scale analysis of the human and mouse transcriptomes. Proc. Natl. Acad. Sci. U.S.A 99:4465-4470.
51. Kloosterboer, F. M., S. A. Luxemburg-Heijs, R. A. van Soest, A. M. Barbui, H. M. van Egmond, M. P. Strijbosch, M. G. Kester, W. A. Marijt, E. Goulmy, R. Willemze, and J. H. Falkenburg. 2004. Direct cloning of leukemia-reactive T cells from patients treated with donor lymphocyte infusion shows a relative dominance of hematopoiesis-restricted minor histocompatibility antigen HA-1 and HA-2 specific T cells. Leukemia 18:798-808.
52. Burrows, S. R., N. Kienzle, A. Winterhalter, M. Bharadwaj, J. D. Altman, and A. Brooks. 2000. Peptide-MHC class I tetrameric complexes display exquisite ligand specificity. J. Immunol. 165:6229-6234.

1-19. (canceled)

20. A peptide for use in the treatment of a malignancy expressing the hECGF-1 protein, wherein
a. the peptide comprises an amino acid sequence encoded by an open reading frame as present in the nucleotide sequence of a transcript of a naturally occurring hECGF-1 allele, wherein the amino acid sequence comprises a polymorphic MHC class I or II minor histocompatibility binding peptide;
b. the amino acid sequence of the MHC binding peptide of a) comprises a polymorphism in amino acids residues 465, 263, 277, 324, 428, 467 and/or 471 in SEQ ID No: 1 due to a single nucleotide polymorphism in the hECGF-1 gene;
c. the reading frame is selected from the amino acid sequences of SEQ ID No: 1 to 4; and
d. the MHC binding peptide is in the context of an MHC class I or MHC class II molecule.

21. A cell for use in the treatment of a malignancy expressing the hECGF-1 protein, wherein
a. the cell is a T lymphocyte comprising a T cell receptor that is capable of interacting with the peptide of claim 20; or
b. the cell is a host cell comprising a nucleic acid molecule encoding the T cell receptor defined in (a).

22. A peptide according to claim 20 or a cell according to claim 21, wherein the subject having a malignancy expressing the hECGF-1 protein has undergone an allogeneic hematopoietic stem cell transplantation.

23. A peptide according to claim 20 or a cell according to claim 21, wherein the malignancy is a hematopoietic malignancy.

24. A peptide according to claim 20 or a cell according to claim 21, wherein the malignancy is a solid tumor present in or originating from a dispensible organ or tissue.

25. A peptide or a cell according to claim 24, wherein the dispensible tissue or organ is selected from the group consisting of testes, kidneys, ovaria, breast, prostate, thyroid, cervix, uterus and pancreas.

26. A peptide or a cell according to claims 24, wherein the malignancy is an at least partly vascularized solid tumor.

27. A peptide comprising an amino acid sequence encoded by an open reading frame as present in the nucleotide sequence of a transcript of a naturally occurring hECGF-1 allele, wherein the amino acid sequence comprises a polymorphic MHC class I or II minor histocompatibility binding peptide, comprising a polymorphism in amino acids residues 465, 263, 277, 324, 428, 467 and/or 471 in SEQ ID No: 1 due to a single nucleotide polymorphism in the hECGF-1 gene and wherein the reading frame is selected from the amino acid sequences of SEQ ID No: 1 to 4.

28. The peptide according to claim 27, wherein the MHC binding peptide is in the context of an MHC class I or MHC class II molecule.

29. A nucleic acid molecule encoding the peptide according to claim 27.

30. A T cell receptor capable of interacting with a peptide as defined in claim 27.

31. A nucleic acid molecule encoding the T cell receptor as defined in claim 30, optionally comprised within a nucleic acid vector.

32. A T lymphocyte comprising a T cell receptor as defined in claim 30.

33. A host cell comprising the nucleic acid molecule as defined in claim 31.

34. A pharmaceutical composition comprising at least one of:
i. an antigenic peptide as defined in claim 27;
ii. a cell as defined in claims 32 or 33;
iii. a gene and/or a vector encoding the peptide as defined in claim 27;
iv. a gene and or a vector encoding a TCR as defined in claim 30;
and at least one pharmaceutically acceptable excipient.

35. A human or humanized antibody specific for a polymorphic hECGF-1 minor histocompatibility antigen, wherein the polymorphic hECGF-1 minor histocompatibility antigen is comprised in a peptide of claim 27, the antigen optionally being in the context of an HLA molecule.

36. The antibody according to claim 35, capable of binding an hECGF-1 mHag encoded by a hECGF-1 nucleotide sequence comprising a SNP at amino acid residues 465, 263, 277, 324, 428, 467 and/or 471 in SEQ ID No: 1, optionally in context of MHC class I or MHC class II molecule.

37. A method for producing a cytotoxic T lymphocyte comprising:
a. bringing undifferentiated lymphocytes into contact with a polymorphic hECGF-1 minor histocompatibility antigen comprising a peptide of claim 27, under conditions conducive of triggering an immune response in vivo or in vitro, or
b. cloning a gene encoding a TCR specific for a polymorphic hECGF-1 minor histocompatibility antigen into a CTL.
